SEQUENCE LISTING (18 sequences)

SEQ ID NO: 1 - DNA, 2625 bp, Artificial Sequence
  Chemically synthesized, Ace2_IgHu_pVax
atggactggacttggaggatcttgttcttggtggctgcagccaccgggacgcacgccaagagtaccatagaggagcaggccaaaaccttcttggacaagttcaaccatgaagctgaagatttgttttaccagtcttctcttgcctcctggaactacaatactaatataaccgaggaaaatgttcagaatatgaacaacgccggggacaaatggtcagcatttctcaaagagcagtctactcttgcgcaaatgtaccccctccaggaaatccagaatctgaccgttaaattacaattgcaggctctgcagcaaaacggatcctcagtcttgtctgaagataagtcaaaacgtctcaataccatattgaataccatgtctactatctactccacgggcaaggtctgcaaccccgacaaccctcaggagtgtctgttgctcgaacccggcctgaatgaaatcatggcgaactctctggattacaatgaacgcctctgggcgtgggagagctggaggtccgaggtgggcaagcagttgcgaccactgtacgaagagtacgtggtgctgaaaaacgaaatggctcgtgcaaatcactatgaggactacggggattactggaggggggattatgaggttaacggtgttgacgggtatgattactctagaggccaactgatcgaagatgtagagcacacgttcgaagagatcaagccactctatgaacacctgcacgcatacgttcgcgccaagctgatgaacgcttacccaagttacatcagcccaattggctgtctgcctgctcatctgctgggggacatgtggggaaggttctggactaatctttacagcctgacagtccctttcggtcagaaaccaaacatcgacgttacggacgctatggtcgatcaagcctgggatgcacagcggatctttaaagaggctgaaaagttttttgtctcagtcggattgcccaacatgacgcagggtttctgggagaatagcatgctgaccgatcctgggaatgtgcagaaagcagtttgccaccccactgcttgggacttgggcaagggtgactttcgcatactcatgtgtaccaaggtgaccatggacgactttttaactgctcatcacgagatgggccacatacagtacgacatggcatacgctgcacagccatttctcctcaggaacggagcaaacgagggcttccacgaggccgtcggcgagatcatgagcttgagtgcagcgactcccaaacatctcaaatctatcggccttctgtctccggactttcaggaagataatgagacagaaataaattttttactgaagcaagcactgaccattgtcggcacccttccgttcacgtacatgctggagaaatggcgttggatggtcttcaaaggagagattcccaaagaccagtggatgaagaaatggtgggagatgaagagagagatagtcggggtcgtggagcccgtgccccatgacgaaacttattgtgatcccgccagtctctttcatgtttccaatgattatagcttcatcaggtactacacacgcacactctatcagttccagtttcaggaagccttatgccaggcagccaagcacgagggcccacttcacaaatgcgacatcagcaatagcacagaagccggacagaaactttttaacatgcttcgtttagggaagagcgaaccatggactttggctctggaaaacgtggtgggcgctaaaaacatgaatgtgcggcccttgctcaattactttgaacctctttttacttggctgaaagaccagaacaagaactctttcgtgggctggagcactgattggtctccatatgctgacgccgctgacccagagcccaagagctgtgacaaaacccatacatgccccccatgccctgcccctgagctgctcggcggcccctccgtgttcttgttccctccgaaaccacccaaggacaccctgatgattagcagaaccccagaggtaacttgcgtcgtggtggatgtctctcatgaggatccggaggttaagttcaattggtatgtggacggcgtggaggtgcataacgctaaaaccaagcctagggaggagcagtacaactccacttatagggtcgtgtccgtgctgacagtgctgcatcaggattggttaaacggaaaagagtacaaatgcaaggtatctaacaaggctctccctgcgcctattgagaagacaatcagtaaagcaaaagggcagcctagagagccccaggtttacacattgccccccagtcgagatgagctgactaaaaatcaagtctcccttacctgcttggttaaagggttttatccatcagacatcgcggtcgagtgggaaagtaatggacagcctgaaaataattacaaaactaccccccccgttcttgatagcgatggctccttcttcctgtacagcaagcttacagtggataaaagtagatggcagcaaggaaacgtttttagttgctcagtgatgcacgaagccctgcataatcattacacccaaaagagtctgagtttgtctccgggcaaaggcggcggcggaggagattatgccgattacgacggaggttattattacgacatggactaatag

SEQ ID NO: 2 - protein, 873 aa, Artificial Sequence
  Chemically synthesized, Ace2_IgHu_pVax
MDWTWRILFLVAAATGTHAKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 3 - DNA, 1185 bp, Artificial Sequence
  Chemically synthesized, minACE2_v1_IgHu_pVax
atggactggacctggatactctttctcgtagcagcagccacacgagtgcattcatccacgatcgaagaacaggctaaaacatttctggacaaatttaatcacgaagccgaagatcttttctatcaaagcagccttgctagttggaactataacaacaatatcacagaggaaaacgtacaaaacatgaacaatgctggggataaatggtctgcattcctgaaagaacaatccacactggctcaaatgtacccacttcaagaaattcaaaatctgacggtgaaactgcaactccaaggcggctccggtagcggtgggtctggtagcggcggttccggaagtggcggttctgtgtgtcatcccaccgcatgggacttggggaagggtgattttcgcattttgatgtgtggtggcgccgctgatccggaacccaagagttgtgataagacccacacctgcccgccttgccctgctcccgaacttttgggcggcccttctgtgtttctctttcccccaaaaccacccaaagacacacttatgataagccggacccccgaagtcacttgtgtggtggttgacgtatctcatgaggacccagaggtcaaatttaattggtacgtggatggcgtcgaagtgcataacgctaaaactaagccccgtgaagagcaatataacagcacatatagggtagtcagtgtcctgacagtgttgcatcaagattggctcaatggaaaggagtacaagtgtaaggtttccaataaagccctgccagcccccattgagaaaactatctcaaaggctaagggacaaccgagagagcctcaggtgtacaccctgccgcccagccgtgacgagcttacaaagaatcaggtaagtttgacgtgcctcgttaaaggcttttatccttccgacattgccgtcgaatgggaatctaatggacagccagagaataactataagaccacaccaccggtcttggattcagatggaagcttcttcttgtatagtaaactcactgtcgacaaaagtcgatggcaacaggggaacgtctttagttgttctgttatgcatgaggcccttcataatcattacacacaaaagtcactttctttgagccctgggaagggtggcggcggcggggattatgctgattacgacggcggatactactacgatatggactagtga

SEQ ID NO: 4 - protein, 393 aa, Artificial Sequence
  Chemically synthesized, minACE2_v1_IgHu_pVax
MDWTWILFLVAAATRVHSSTIEEQAKTFLDKFNHEAEDLFYQSSLASWNYNNNITEENVQNMNNAGDKWSAFLKEQSTLAQMYPLQEIQNLTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 5 - DNA, 1173 bp, Artificial Sequence
  Chemically synthesized, minACE2_v2_IgHu_pVax
atggactggacctggatactctttctcgtagcagcagccacacgagtgcattcaagcaccattgaggaacaagccaaaacattcctggataagttcaaccatgaagcggaagatcttttctaccaaagttcacttgcttcctggaattacaataataacatcacagaagaaaacgtgcagaacatgaataatgccggcgacaaatggtccgcctttctcaaggagcaaagcactcttgcgcaaatgtacccactgcaagagatccagaaccttacagtaaaattgcaactccaaggtgggtccggatctggcggcagtggttccggcggttcaggaagcggtggtagttgtactgcctgggatctgggcaagggcgattttcgcatcctgtgcggcggagcagcggacccggagcccaaaagttgtgataagacccacacctgtcccccatgtcctgccccagagcttttgggtggtcctagcgtgttccttttcccgccgaaaccgccaaaggacacacttatgatctctcgtacccccgaagtcacatgtgttgtcgtagacgtgtcccacgaggacccagaagtgaaatttaactggtatgtcgacggcgtggaagtacacaatgcgaaaactaaaccaagggaagaacaatataattccacatatcgtgtggtaagtgttctcacagtcctgcatcaggactggctgaacgggaaggaatacaagtgtaaggttagtaacaaagctcttccggcccccattgaaaagaccatctccaaagcgaaaggccaacccagagaaccgcaggtgtacactttgccgcccagcagagacgagttgactaagaatcaagtctcactcacttgcctggtcaagggcttctatccttccgacattgcagttgaatgggaatccaacggacaaccggaaaataactataaaacaacaccaccggtgctcgatagtgacggatctttcttcctttatagcaaactcaccgttgacaagtcccgctggcagcaggggaacgtcttttcctgtagtgtgatgcacgaggctcttcacaatcattacacgcagaaatctctgagtctcagtcccgggaaaggtggggggggcggagattatgcggactacgatggcgggtactactacgatatggattgatag

SEQ ID NO: 6 - protein, 389 aa, Artificial Sequence
  Chemically synthesized, minACE2_v2_IgHu_pVax
MDWTWILFLVAAATRVHSSTIEEQAKTFLDKFNHEAEDLFYQSSLASWNYNNNITEENVQNMNNAGDKWSAFLKEQSTLAQMYPLQEIQNLTVKLQLQGGSGSGGSGSGGSGSGGSCTAWDLGKGDFRILCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 7 - DNA, 1185 bp, Artificial Sequence
  Chemically synthesized, minACE2_v3_IgHu_pVax
atggactggacctggatactctttctcgtagcagcagccacacgagtgcattcatcaaccattgaagaacaggccaaaactttcctcgacaagtttaatcacgaggcagaagatctgttttatcaaagttccctcgcgagctggaactacaataacaatatcacagaagaaaacgtacagaatatgaataacgcaggcgataaatggtctgcatttctcaaagaacaatctacgctggcgcaaatgtaccctctccaagagatacaagcccttaccgttaagctccagctgcaaggcggctccggttccggcggtagcggctctggtggtagcgggagcggtggatctgtgtgtcatccaactgcatgggacctgggaaaaggggattttcggattctcatgtgcggtggtgccgccgacccagagcccaagtcttgtgacaagacccatacttgcccaccctgtcctgctcccgaactgctgggtggcccttctgtattccttttcccaccaaaaccacctaaggatactcttatgatcagcagaacacccgaagtaacatgtgtggtggtcgatgtgtctcacgaagatccagaagtgaagtttaattggtatgtcgatggcgtggaagtacataatgccaaaacgaagccccgggaagaacagtataattctacctatagagtggtttctgtgttgaccgtgctccatcaggattggctgaacggtaaggaatacaagtgtaaggtttctaataaggcactccctgccccgatagagaaaacaatttccaaggctaaggggcaacccagagagccacaagtctataccttgcccccatcacgggacgaacttacgaagaatcaagtatcccttacctgcctggtcaaaggattttatcccagtgatatagccgttgaatgggagtctaacggccaacccgagaataattataagacaacaccgccggtattggactcagatggctccttctttctgtacagcaagctgacggtagacaagagccgctggcaacaagggaacgtctttagttgttctgtcatgcacgaagcccttcacaatcactatacacagaaatctctgtctttgagtcccgggaaaggaggaggaggaggagactatgctgattatgatggcggatattactatgatatggattaatag

SEQ ID NO: 8 - protein, 393 aa, Artificial Sequence
  Chemically synthesized, minACE2_v3_IgHu_pVax
MDWTWILFLVAAATRVHSSTIEEQAKTFLDKFNHEAEDLFYQSSLASWNYNNNITEENVQNMNNAGDKWSAFLKEQSTLAQMYPLQEIQALTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 9 - DNA, 1173 bp, Artificial Sequence
  Chemically synthesized, minACE2_v4_IgHu_pVax
atggactggacctggatactctttctcgtagcagcagccacacgagtgcattcaagcacgatagaggagcaagccaaaaccttccttgataagtttaatcatgaggcagaggatctgttctaccaaagctccctcgcttcctggaattataataataacattaccgaagagaatgttcaaaacatgaataatgctggggataagtggtcagccttcctcaaagaacaatccaccctggcccaaatgtatcctctgcaagagattcaagccctgacagtcaaactccagctgcaaggcggcagcggctccggtggcagtggatcaggcggatcaggctcaggcggctcttgtactgcatgggatcttggcaagggtgatttcagaatcctttgtggcggagctgcggaccccgagcctaagtcctgcgataaaacccatacatgcccgccatgccctgccccagagttgctgggtgggccgagcgtgtttctcttcccacccaaacccccaaaagacacactgatgatatcccgtacgccagaagtgacttgtgttgtcgttgacgtgtcacatgaagatcctgaagtgaaatttaattggtatgttgatggcgtcgaggtacataacgcgaaaacaaaacccagggaagagcaatacaattccacatacagagttgtttctgtgttgactgtgttgcatcaggattggctcaatggcaaggaatacaagtgcaaggtctccaataaagctctccctgcccccattgaaaagacaatatctaaagccaaggggcaacctcgcgaaccgcaggtgtacaccttgcctccctccagagacgaactgaccaagaatcaagttagtttgacgtgtcttgtgaagggattctatccctcagatatcgcggtcgaatgggagagtaatgggcaaccggaaaataactataagacaaccccgcccgtcctcgatagcgatggatcattcttcctgtatagcaagctgactgtggacaaaagcagatggcaacaaggtaacgtcttctcatgttcagttatgcacgaggcgctccacaatcattatacccagaaaagtttgtctctcagtccaggcaaaggtggtggtggtggtgattacgctgactacgacggtggttactattacgatatggactaatga

SEQ ID NO: 10 - protein, 389 aa, Artificial Sequence
  Chemically synthesized, minACE2_v4_IgHu_pVax
MDWTWILFLVAAATRVHSSTIEEQAKTFLDKFNHEAEDLFYQSSLASWNYNNNITEENVQNMNNAGDKWSAFLKEQSTLAQMYPLQEIQALTVKLQLQGGSGSGGSGSGGSGSGGSCTAWDLGKGDFRILCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 11 - DNA, 1185 bp, Artificial Sequence
  Chemically synthesized, minACE2_v5_IgHu_pVax
atggactggacctggatactctttctcgtagcagcagccacacgagtgcattcatctaccattgaagaacaggcgaaatatttcttggacaagttcaatgtcgaagctgaggacttgttttataagagtagccttgcttcttggaattacaataacaacataacagaagagaacgttcagaatatgaacaacgccggcgacaaatggagtgcatttctcaaagaacaatccacgttggcacaaatgtatccgttgcaggaaatacaagctctgaccgtcaagcttcaacttcaaggcggctctgggtccggtggcagtgggtctggtggatctgggtcaggcggctcagtttgccatcctacggcctgggacctcgggaaaggggattttcggatacttatgtgtggtggggcagccgatcctgagcctaagtcctgcgataagactcacacgtgtccaccatgcccggctcccgaactcctcggtggcccatcagtgttcctctttccacctaagccacctaaagacacgcttatgatttctcgcactccagaagtgacatgtgttgttgtagacgttagtcacgaagaccctgaggtgaagtttaattggtacgtggacggagtggaagttcataatgccaagactaagcctagagaagagcagtacaattctacctacagggtggtaagcgttctcacggttctccatcaagactggcttaatggaaaagagtataaatgcaaagtgagtaacaaggctcttcctgccccaattgagaagaccattagtaaagcgaaaggtcagcctagagagccgcaggtctacactctgccacctagtcgagatgaactcactaagaatcaagtcagtttgacatgtctcgtaaagggcttctacccaagtgatatcgcggtggagtgggagtccaacggtcagccagaaaacaactacaagaccacaccccctgtgctcgattcagatggctcattctttctctacagtaagctgacagttgataaaagtagatggcaacaaggcaacgtcttttcttgctctgtcatgcatgaagcccttcacaaccattatacacaaaagtcattgagcctgagtcctggcaagggaggaggcggcggcgattatgccgattacgacggcggttactattacgacatggattagtaa

SEQ ID NO: 12 - protein, 393 aa, Artificial Sequence
  Chemically synthesized, minACE2_v5_IgHu_pVax
MDWTWILFLVAAATRVHSSTIEEQAKYFLDKFNVEAEDLFYKSSLASWNYNNNITEENVQNMNNAGDKWSAFLKEQSTLAQMYPLQEIQALTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 13 - DNA, 1185 bp, Artificial Sequence
  Chemically synthesized, minACE2_v6_IgHu_pVax
atggactggacctggatactctttctcgtagcagcagccacacgagtgcattcatccactatagaggaacaagcgaaatacttcctggacaagttcaatgcggaagccgaagacctgttttatatgtcatctctggccagctggaactataacaacaacattacggaagagaacgtgcaaaacatgaacaatgccggcgacaaatggtctgcattcctgaaagaacaatccacccttgcacaaatgtaccctctccaagaaatacaagcactgacagtcaagctgcagctgcaaggtggatctggttctggcggaagcggatctggtggcagtgggagtggtggttccgtatgtcatcctacagcctgggatctcgggaaaggggatttccgtatccttatgtgcggcggggcagccgatcctgagccaaaatcctgcgacaaaacacatacatgtccaccctgtccggcccccgagcttctgggtggtccatctgtattcctgtttccccctaaaccacctaaggataccctcatgatcagccggacccccgaagtcacatgtgtggtggtggacgtgtcacacgaagacccggaagtcaaattcaattggtacgtcgatggagtcgaagtacataatgccaaaactaaaccgcgagaggagcagtataacagtacttatcgcgtggtcagtgtcctgaccgtcctgcaccaagattggctgaatggaaaagagtataaatgcaaggtaagcaacaaagccctgccagcgccaattgagaaaactatcagcaaagccaagggacagccaagagaaccacaagtgtacaccttgccgcccagtagggatgaactcacgaagaatcaagtgagcttgacctgcttggtcaaaggcttttatccttcagacatagcggtggagtgggaatccaacggccagcccgaaaataactataaaactaccccacctgtacttgatagtgacggcagcttcttcttgtactccaagctgaccgtggataaatctcgatggcaacaagggaacgtcttcagctgttcagtaatgcatgaggcactgcataatcactatacacaaaagagtctctctttgtccccaggaaagggtggtggaggaggtgattacgccgactacgatggcgggtattattatgatatggattgataa

SEQ ID NO: 14 - protein, 393 aa, Artificial Sequence
  Chemically synthesized, minACE2_v6_IgHu_pVax
MDWTWILFLVAAATRVHSSTIEEQAKYFLDKFNAEAEDLFYMSSLASWNYNNNITEENVQNMNNAGDKWSAFLKEQSTLAQMYPLQEIQALTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 15 - DNA, 1185 bp, Artificial Sequence
  Chemically synthesized, minACE2_v7_IgHu_pVax
atggactggacctggatactctttctcgtagcagcagccacacgagtgcattcaagtaccatcgaagagcaagctaaactgttcctggataaattcaatgtcgaggccgaagatctcttctatatgtcatccctcgcttcctggaattataataacaatattactgaagagaatgtacagaacatgaataatgccggagataaatggtccgcttttctgaaggagcaatccacacttgcccaaatgtatccgctccaagaaatacaagcgcttacggttaaactccaactgcaaggtggctctggctcaggcggctctggaagcggtggctcagggtcaggtggctccgtgtgtcacccaaccgcatgggatctgggcaagggagattttcgaattcttatgtgcggcggcgctgcagaccctgaacccaaaagctgtgataaaactcacacctgcccgccatgtccagcacccgaactcttgggcggccccagtgtcttccttttcccaccaaaaccccctaaggatactctgatgatctcacggactccagaggtaacctgtgtggtggtcgacgtgtcacacgaggacccggaagttaaatttaattggtacgtcgatggagttgaggtgcataatgctaaaactaaacctcgggaagaacagtacaattcaacttatcgagtcgtgagtgtgctcactgtccttcaccaagactggttgaacgggaaggaatacaagtgcaaagtcagtaacaaagcgctccccgcccctatcgagaagactatctctaaggccaaagggcaacccagggagcctcaggtttatacgctgccaccatcccgggatgaactgaccaagaaccaagtaagcttgacctgtcttgtgaagggtttctatccttccgatattgcggtagaatgggaaagcaatggacaaccggaaaataattataaaaccactccgcccgttcttgattccgacggatcattctttctgtatagcaaactgaccgtggataagtctagatggcagcaagggaacgtgttcagttgctctgtaatgcacgaagcattgcataatcattacacgcaaaagtccctctctttgagccccggcaagggaggaggaggaggagactatgctgattatgacggtggctactactatgacatggactaatag

SEQ ID NO: 16 - protein, 393 aa, Artificial Sequence
  Chemically synthesized, minACE2_v7_IgHu_pVax
MDWTWILFLVAAATRVHSSTIEEQAKLFLDKFNVEAEDLFYMSSLASWNYNNNITEENVQNMNNAGDKWSAFLKEQSTLAQMYPLQEIQALTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

SEQ ID NO: 17 - DNA, 1185 bp, Artificial Sequence
  Chemically synthesized, minACE2_v8_IgHu_pVax
atggactggacctggatactctttctcgtagcagcagccacacgagtgcattcatcaactatagaagaacaagccaaactgtttctcgacaagttcaatgctgaagcagaagatctgttctataaatcttcccttgcgtcatggaattacaacaataacattacggaagaaaacgtccagaacatgaacaatgctggcgacaaatggtctgcatttctgaaagaacagtctacattggcgcagatgtatccattgcaggagatacaggctctcactgtaaagctccaactgcagggtggaagcggctccggtgggtccggcagtggcggtagtggaagtggtggttccgtctgccacccaacagcttgggatttgggtaaaggggactttaggatacttatgtgtggcggggctgccgacccagagcctaagtcctgtgataaaacgcacacctgcccgccctgtcccgcacccgagctgctgggtggcccatcagtattcctgtttccacccaagcccccgaaggatactctgatgatttcacgtactcccgaagtaacttgtgtggtagtcgatgtgtcacacgaagatcccgaggtgaaatttaactggtacgtggacggggttgaggtgcacaatgcgaaaactaagccccgggaagagcagtataactctacttaccgagtggtgagcgtcctcaccgtgctccaccaagactggctgaacggcaaggagtataaatgtaaggttagcaataaggccctgccagctccaattgagaaaaccatcagcaaggccaaagggcagccccgcgagccccaggtgtacacgctgccgcctagcagagatgagctgactaagaatcaggtctctcttacctgtttggtgaagggcttttaccctagcgacattgccgtggaatgggagtccaacggccagcctgaaaacaattacaagacaactcccccggtcttggatagtgacgggagcttcttcctttactccaagctgacagtcgacaagtctagatggcagcaagggaacgtcttcagctgtagcgttatgcacgaggccctgcacaatcattatacgcagaaatctctttctttgtctcccggtaagggggggggcggcggggattatgctgattatgatggtggctattattatgatatggactaatga

SEQ ID NO: 18 - protein, 393 aa, Artificial Sequence
  Chemically synthesized, minACE2_v8_IgHu_pVax
MDWTWILFLVAAATRVHSSTIEEQAKLFLDKFNAEAEDLFYKSSLASWNYNNNITEENVQNMNNAGDKWSAFLKEQSTLAQMYPLQEIQALTVKLQLQGGSGSGGSGSGGSGSGGSVCHPTAWDLGKGDFRILMCGGAADPEPKSCDKTHTCPPCPAPELLGGPSVFLFPPKPPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGDYADYDGGYYYDMD

(19) United States
(12) Patent Application Publication
(10) Pub. No.: US 2023/0137756 A1
(43) Pub. Date: May 4, 2023

(54) Synthetic Soluble Receptor Mimics and Methods of Use for Treatment of COVID-19

(71) Applicant: The Wistar Institute of Anatomy and Biology, Philadelphia, PA (US)

(72) Inventor: Daniel Kulp, Moorestown, NJ (US)

(21) Appl. No.: 17/907,697

(22) PCT Filed: Mar. 30, 2021
(86) PCT No.: PCT/US21/24786

§ 371 Date: Sep. 29, 2022

Related U.S. Application Data
(60) Provisional application No. 63/001,882, filed on Mar. 30, 2020.

(51) Int. Cl. C07K 14/705; A61P 31/14

(52) U.S. Cl. CPC C07K 14/705; A61P 31/14; C07K 2319/30; A61K 38/00

(57) ABSTRACT

The present invention provides compositions comprising cellular receptor mimics and methods for treating or preventing coronavirus infection or a disease or disorder associated with coronavirus infection such as COVID-19.

### CROSS REFERENCE TO RELATED APPLICATIONS

This application claims priority to U.S. Provisional Application No. 63/001,882, filed Mar. 30, 2021, which is hereby incorporated by reference herein in its entirety.

### BACKGROUND OF THE INVENTION

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, 0C43, NL63, and HKU1 are endemic in the human population. In 2019, a novel coronavirus emerged in China and is now known as SARS-CoV-2, and is associated with the development of Coronavirus Disease 2019 (COVID-19).

Preventative delivery of recombinant mAb for infectious diseases is an incredibly sluggish process facing hurdles including development limitations, bioprocess manufacturing, stability, and IV delivery requiring hours plus follow-up. This is extremely challenging for deployable personnel and large populations.

There is an extremely imminent need for new therapeutics to combat the alarming spread of new coronaviruses. The present invention satisfies this unmet need.

### BRIEF DESCRIPTION OF THE DRAWINGS

FIG. 1 depicts an overview of the rapid DMAb platform.

FIG. 2 depicts soluble receptor mimic concepts.

FIG. 3A through FIG. 3B depict ACE2-human-Fc binding to SARS-CoV-2. (FIG. 3A) SPR binding sensorgrams capturing CoV2-S1-His on a NTA-chip and flowing Ace2-human-Fc as an analyte. (FIG. 4B) SARS-CoV2-2 S1 protein captured with anti-His antibody and Ace2-human-Fc binding detected by anti-human secondary.

FIG. 4A through FIG. 4D depict DMAb characterization of existing DMAbs and in vivo expression profiles. (FIG. 4A) In vivo expression for DMAbs as one or two plasmids. (FIG. 4B) Coverage of VH and VL genes produced as DMAbs. (FIG. 4C) CDRH3 lengths vs in vivo expression levels. (FIG. 4D) Unique DMAb HC genes vs in vivo expression levels.

### BRIEF SUMMARY OF THE INVENTION

In one embodiment, the invention relates to a composition comprising a nucleic acid molecule encoding at least one synthetic cellular receptor peptide which specifically binds to a coronavirus Spike antigen, or a fragment thereof. In one embodiment, the synthetic cellular receptor peptide is ACE2, CD147, CD107a, CD13, GRP78 and DPP4, or fragment thereof, or a variant thereof.

In one embodiment, the synthetic cellular receptor peptide is fused to an antibody Fc domain.

In one embodiment, two or more synthetic cellular receptor peptides form a multimeric complex.

In one embodiment, the nucleic acid molecule comprises a nucleotide sequence that encodes a peptide comprising an amino acid sequence having at least about 90% identity over an entire length of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleic acid molecule comprises a nucleotide sequence that encodes a peptide fragment comprising at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleotide sequence encodes a peptide comprising an amino acid sequence having at least about 90% identity to at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleic acid molecule encodes an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the nucleic acid molecule comprises a nucleotide sequence having at least about 90% identity over an entire length of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises a nucleotide sequence comprising at least 30% of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises a nucleotide sequence comprising at least about 90% identity to at least 30% of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

In one embodiment, the nucleic acid molecule comprises an expression vector. In one embodiment, the nucleic acid molecule is incorporated into a viral particle.

In one embodiment, the composition further comprises a pharmaceutically acceptable excipient. In one embodiment, the composition further comprises an adjuvant.

In one embodiment, the invention relates to a composition comprising at least one synthetic cellular receptor peptide which specifically binds to a coronavirus Spike antigen, or a fragment thereof. In one embodiment, the synthetic cellular receptor peptide is ACE2, CD147, CD107a, CD13, GRP78 or DPP4, a fragment thereof, or a variant thereof.

In one embodiment, the synthetic cellular receptor peptide is fused to an antibody Fc domain.

In one embodiment, two or more synthetic cellular receptor peptides form a multimeric complex.

In one embodiment, the synthetic cellular receptor peptide comprises an amino acid sequence having at least about 90% identity over an entire length of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the synthetic cellular receptor peptide comprises a peptide fragment comprising at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the synthetic cellular receptor peptide comprises an amino acid sequence having at least about 90% identity to at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the synthetic cellular receptor peptide comprises an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the composition further comprises a pharmaceutically acceptable excipient. In one embodiment, the composition further comprises an adjuvant.

In one embodiment, the invention relates to a nucleic acid molecule comprising a nucleotide sequence encoding at least one synthetic cellular receptor peptide which specifically binds to a coronavirus Spike antigen, or a fragment thereof.

In one embodiment, the synthetic cellular receptor peptide is ACE2, CD147, CD107a, CD13, GRP78 and DPP4, or fragment thereof, or a variant thereof.

In one embodiment, the synthetic cellular receptor peptide is fused to an antibody Fc domain.

In one embodiment, two or more synthetic cellular receptor peptides form a multimeric complex.

In one embodiment, the nucleic acid molecule comprises a nucleotide sequence that encodes a peptide comprising an amino acid sequence having at least about 90% identity over an entire length of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleic acid molecule comprises a nucleotide sequence that encodes a peptide fragment comprising at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleotide sequence encodes a peptide comprising an amino acid sequence having at least about 90% identity to at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the nucleic acid molecule encodes an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the nucleic acid molecule comprises a nucleotide sequence having at least about 90% identity over an entire length of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises a nucleotide sequence comprising at least 30% of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises a nucleotide sequence comprising at least about 90% identity to at least 30% of the nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In one embodiment, the nucleic acid molecule comprises SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

In one embodiment, the nucleic acid molecule comprises an expression vector. In one embodiment, the nucleic acid molecule is incorporated into a viral particle.

In one embodiment, the invention relates to a synthetic cellular receptor peptide which specifically binds to a coronavirus Spike antigen, or a fragment thereof. In one embodiment, the synthetic cellular receptor peptide is ACE2, CD147, CD107a, CD13, GRP78 or DPP4, a fragment thereof, or a variant thereof.

In one embodiment, the synthetic cellular receptor peptide is fused to an antibody Fc domain.

In one embodiment, two or more synthetic cellular receptor peptides form a multimeric complex.

In one embodiment, the synthetic cellular receptor peptide comprises an amino acid sequence having at least about 90% identity over an entire length of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the synthetic cellular receptor peptide comprises a peptide fragment comprising at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In one embodiment, the synthetic cellular receptor peptide comprises an amino acid sequence having at least about 90% identity to at least 30% of the amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the synthetic cellular receptor peptide comprises an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In one embodiment, the invention relates to a method of treating or preventing a disease or disorder associated with a coronavirus infection in a subject in need thereof, the method comprising administering a composition comprising a nucleic acid molecule encoding at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of treating or preventing a disease or disorder associated with a coronavirus infection in a subject in need thereof, the method comprising administering a composition comprising at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of treating or preventing a disease or disorder associated with a coronavirus infection in a subject in need thereof, the method comprising administering a nucleic acid molecule encoding at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of treating or preventing a disease or disorder associated with a coronavirus infection in a subject in need thereof, the method comprising administering at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of protecting a subject in need thereof from a disease or disorder associated with coronavirus, the method comprising administering a composition comprising a nucleic acid molecule encoding at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of protecting a subject in need thereof from a disease or disorder associated with coronavirus, the method comprising administering a composition comprising at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of protecting a subject in need thereof from a disease or disorder associated with coronavirus, the method comprising administering a nucleic acid molecule encoding at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

In one embodiment, the invention relates to a method of protecting a subject in need thereof from a disease or disorder associated with coronavirus, the method comprising administering at least one synthetic cellular receptor peptide which specifically binds to a coronavirus Spike antigen, or a fragment thereof. In one embodiment, the disease or disorder associated with a coronavirus is selected from the group consisting of SARS, 229E, NL63, OC43, HKU1, MERS and SARS-CoV-2. In one embodiment, the disease or disorder is COVID-19. In one embodiment, the composition is administered by way of injection, electroporation, or a combination thereof.

### DETAILED DESCRIPTION

This invention directly addresses the need for preventative medical countermeasures against COVID-19. DMAbs have the potential to be delivered as pre-exposure prophylaxis to at-risk populations such as the warfighter and other military personnel, health-care workers, and other at-risk or vulnerable populations such as the elderly. In addition to prevention, DMAbs may be delivered as a treatment option for potentially exposed individuals. DMAbs are a critical platform for three key reasons: 1) they can be rapidly deployed to the population as mass production of clean DNA is extremely routine, 2) there is a long track record of delivering DNA safely to people and 3) pathways to the clinic for DNA-based therapeutics are established.

The present invention provides compositions comprising synthetic soluble receptor immunotherapeutics. In some embodiments, the receptor comprises an ACE2 receptor, CD147, CD107a, CD13, GRP78, DPP4 fragments thereof, variants thereof, or any combination thereof. The present invention can be used to treat or prevent diseases or disorders which require receptor interaction for infection including, but not limited to, coronaviruses such as SARS, 229E, NL63, OC43, HKU1, MERS and SARS-CoV-2 (COVID-19).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antigen” refers to proteins that have the ability to generate an immune response in a host. An antigen may be recognized and bound by an antibody. An antigen may originate from within the body or from the external environment.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883. “Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Adjuvant” as used herein means any molecule added to the vaccine described herein to enhance the immunogenicity of the antigen.

“Coding sequence” or “encoding nucleic acid” as used herein may refer to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may have Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” of a compound is that amount of compound which is sufficient to provide an effect to the subject or system to which the compound is administered.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

The terms “patient,” “subject,” “individual,” and the like are used interchangeably herein, and refer to any animal, or cells thereof whether in vitro or in situ, amenable to the methods described herein. In some embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter. “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein may facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances.

Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C. “Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

A “therapeutic” treatment is a treatment administered to a subject who exhibits signs or symptoms of a disease or disorder, for the purpose of diminishing or eliminating the frequency or severity of those signs or symptoms.

As used herein, “treating a disease or disorder” means reducing the frequency or severity, or both, of at least one sign or symptom of the disease or disorder experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder, including alleviating signs and/or symptoms of such diseases and disorders.

To “treat” a disease or disorder as the term is used herein, means to reduce the frequency or severity of at least one sign or symptom of a disease or disorder experienced by a subject.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in part upon the discovery that the coronavirus spike antigen, including the spike antigen from SARS and SARS-CoV-2 mediates infection through interaction with cellular receptors including, but not limited to, ACE2, CD147, CD107a, CD13, GRP78 and DPP4.

In one embodiment, the invention provides compositions comprising a soluble receptor mimic or a nucleic acid molecule encoding the same. In some embodiments, the composition mimics a receptor used for viral entry into cells. Therefore in some embodiments, the soluble receptor mimic functions as an alternative interacting partner to cellular receptors for viral particle binding, thus serving as a decoy cellular receptor.

Therefore in some embodiments, the invention provides compositions comprising one or more soluble receptor mimic of ACE2, CD147, CD107a, CD13, GRP78 and DPP4, a fragment thereof, or a variant thereof, or a nucleic acid molecule encoding the same. In some embodiments, the invention provides fusion molecules comprising ACE2, CD147, CD107a, CD13, GRP78 and DPP4, a fragment thereof, or a variant thereof fused to an antibody Fc domain to create an antibody-like biologic, or a nucleic acid molecule encoding the same.

In some embodiments, the invention provides methods of treating or preventing coronavirus infection or a disease or disorder of a coronavirus coronavirus infection comprising administering to a subject or a soluble receptor mimic of ACE2, CD147, CD107a, CD13, GRP78 or DPP4, a fragment thereof, or a variant thereof, or a nucleic acid molecule encoding the same. In some embodiments, the methods include administration of antibody-like biologic fusion molecules comprising ACE2, CD147, CD107a, CD13, GRP78 or DPP4, a fragment thereof, or a variant thereof fused to an antibody Fc domain, or a nucleic acid molecule encoding the same.

### Compositions

Provided herein are immunogenic compositions, such as vaccines, comprising an antibody-like molecule which specifically binds to a Coronavirus disease 2019 (COVID-19) antigen, a fragment thereof, a variant thereof, or a combination thereof. The vaccine can be used to protect against any number of strains of coronavirus, including SARS, 229E, NL63, OC43, HKU1, MERS and SARS-CoV-2 and SARS-CoV-2, thereby treating, preventing, and/or protecting against coronavirus based pathologies, such as COVID-19.

In one embodiment, the present invention provides compositions for mimicking a cellular receptor. In one embodiment, the present invention provides compositions for specifically binding to a coronavirus Spike protein, preventing its interaction with a cellular receptor. In some embodiments, the composition mimics ACE2, CD147, CD107a, CD13, GRP78 or DPP4 for the purposes of binding to and inhibiting a coronavirus spike protein.

One of skill in the art will appreciate that compositions for mimicking a cellular receptor can be administered as a protein, a nucleic acid construct encoding a protein, or combinations thereof. Numerous vectors and other compositions and methods are well known for administering a protein or a nucleic acid construct encoding a protein to cells or tissues. Therefore, the invention includes soluble proteins that mimic cellular receptors as well as nucleic acid molecules encoding soluble proteins that mimic cellular receptors.

The vaccine can be a DNA vaccine, a peptide vaccine, or a combination DNA and peptide vaccine. The DNA vaccine can include a nucleic acid sequence encoding ACE2, CD147, CD107a, CD13, GRP78 or DPP4, or a fragment or variant thereof. The nucleic acid sequence can be DNA, RNA, cDNA, a variant thereof, a fragment thereof, or a combination thereof. The nucleic acid sequence can also include additional sequences that encode linker, leader, or tag sequences that are linked to the sequence encoding ACE2, CD147, CD107a, CD13, GRP78 or DPP4 by a peptide bond. The peptide vaccine can include an ACE2, CD147, CD107a, CD13, GRP78 or DPP4 peptide, a ACE2, CD147, CD107a, CD13, GRP78 or DPP4 protein, a variant thereof, a fragment thereof, or a combination thereof.

One of skill in the art will appreciate that an ACE2, CD147, CD107a, CD13, GRP78 or DPP4 polypeptide, a recombinant ACE2, CD147, CD107a, CD13, GRP78 or DPP4 polypeptide, an ACE2, CD147, CD107a, CD13, GRP78 or DPP4 polypeptide fragment, a variant thereof, a fusion thereof, or a nucleic acid molecule encoding any of the same can be administered singly or in any combination.

In some embodiments, the protein or peptide comprises an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In some embodiments, the protein or peptide comprises a fragment comprising at least 20%, 30%, 40%, 50%, 60%, 65%, 70%, 75%, 80%, 85%, 90%, 91%, 92% 93%, 94%, 95%, 96%, 97%, 98%, or 99% of the full length of a protein or peptide comprising an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In some embodiments, the protein or peptide comprises a polypeptide having substantial homology to a protein or peptide disclosed herein. In some embodiments, the polypeptide has at least 75%, 80%, 85%, 90%, 91%, 92% 93%, 94%, 95%, 96%, 97%, 98%, or 99% sequence identity with SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

A protein of the invention may be synthesized by conventional techniques. For example, the proteins may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a polypeptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or protein of the invention, conjugated with at least one other molecule, may be prepared by fusing, through recombinant techniques, the N-terminal or C-terminal end of the peptide or protein, and the sequence of a selected protein or selectable marker with a desired biological function. The resultant fusion proteins contain the peptide of the invention fused to the selected protein or marker protein as described herein. Examples of proteins which may be used to prepare fusion proteins include immunoglobulins and regions thereof, glutathione-S-transferase (GST), hemagglutinin (HA), and truncated myc. A protein of the invention may be developed using a biological expression system. The use of these systems allows the production of large libraries of random sequences and the screening of these libraries for sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The protein of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The present invention further encompasses fusion proteins in which the protein of the invention or fragments thereof, are recombinantly fused or chemically conjugated (including both covalent and non-covalent conjugations) to heterologous proteins (i.e., an unrelated protein or portion thereof, e.g., at least 10, at least 20, at least 30, at least 40, at least 50, at least 60, at least 70, at least 80, at least 90 or at least 100 amino acids of the polypeptide) to generate fusion proteins. The fusion does not necessarily need to be direct but may occur through linker sequences.

In one example, a fusion protein in which a protein of the invention or a fragment thereof can be fused to sequences derived from various types of immunoglobulins. For example, a polypeptide of the invention can be fused to a constant region (e.g., hinge, CH2, and CH3 domains) of human IgG or IgM molecule, for example, as described herein, so as to make the fused protein or fragments thereof more soluble and stable in vivo. In another embodiment, such fusion proteins can be administered to a subject so as to inhibit interactions between a ligand and its receptors in vivo. Such inhibition of the interaction will block or suppress signal transduction which triggers certain cellular responses.

In one embodiment, the peptide comprises a domain that enhances stability or half-life of the fusion protein. For example, in one embodiment, the domain comprises at least one region of an immunoglobulin, human serum albumin (HSA), or a peptide or antibody fragment that binds to immunoglobulin, HSA, the erythrocyte cell surface, or the neonatal Fc receptor. In one embodiment, the domain comprises a fragment or variant of at least one region of an immunoglobulin. For example, in one embodiment, the domain comprises an Fc region of an immunoglobulin. Exemplary immunoglobulins include, but is not limited to, IgG1, IgG2, IgG3, IgG4, IgM, IgA, IgE, and IgD.

In one aspect, the fusion protein comprises a polypeptide of the invention which is fused to a heterologous signal sequence at its N-terminus. For example, the signal sequence naturally found in the protein of the invention can be replaced by a signal sequence which is derived from a heterologous origin. Various signal sequences are commercially available. For example, the secretory sequences of melittin and human placental alkaline phosphatase (Stratagene; La Jolla, Calif.) are available as eukaryotic heterologous signal sequences. As examples of prokaryotic heterologous signal sequences, the phoA secretory signal (Sambrook, et al., supra; and Current Protocols in Molecular Biology, 1992, Ausubel, et al., eds., John Wiley & Sons) and the protein A secretory signal (Pharmacia Biotech; Piscataway, N.J.) can be listed. Another example is the gp67 secretory sequence of the baculovirus envelope protein (Current Protocols in Molecular Biology, 1992, Ausubel, et al., eds., John Wiley & Sons).

In another embodiment, a protein of the invention can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz, et al., 1989, Proc. Natl. Acad. Sci. USA 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson, et al., 1984, Cell 37:767) and the “flag” tag (Knappik, et al., 1994, Biotechniques 17(4):754-761). These tags are especially useful for purification of recombinantly produced proteins of the invention.

### Antibody-Like Fusion Molecules

In one embodiment, the invention provides a composition comprising a synthetic antibody-like biologic comprising a soluble receptor polypeptide fragment fused to an antibody or fragment thereof. In one embodiment, the composition comprises a nucleotide sequence encoding a synthetic antibody. The composition, when administered to a subject in need thereof, can result in the generation of a synthetic antibody-like biologic in the subject. The synthetic antibody-like biologic can bind a target molecule (i.e., viral particle) present in the subject. Such binding can prevent the target viral particle from binding to a cellular receptor.

In various embodiments, the synthetic antibody-like biologic comprises an amino acid sequence of SEQ ID NO: 2.

Further, the invention encompasses a polypeptide having substantial homology to a protein or peptide disclosed herein. In some embodiments, the synthetic antibody-like biologic comprises an amino acid sequence having at least 75%, 80%, 85%, 90%, 91%, 92% 93%, 94%, 95%, 96%, 97%, 98%, or 99% sequence identity with SEQ ID NO: 2.

In certain embodiments, the composition can treat, prevent, and/or protect against a viral infection. In certain embodiments, the composition can treat, prevent, and/or protect against a coronavirus infection. In certain embodiments, the coronavirus is SARS or SARA-CoV2.

### ScFv Antibody

In one embodiment, the synthetic antibody-like biologic comprises ScFv DMAb. In one embodiment, ScFv DMAb relates to a Fab fragment without the of CH1 and CL regions. Thus, in one embodiment, the ScFv DMAb relates to a Fab fragment DMAb comprising the VH and VL. In one embodiment, the ScFv DMAb comprises a linker between VH and VL. In one embodiment, the ScFv DMAb is an ScFv-Fc DMAb. In one embodiment, the ScFv-Fc DMAb comprises the VH, VL and the CH2 and CH3 regions. In one embodiment, the ScFv-Fc DMAb comprises a linker between VH and VL. In one embodiment, the ScFv DMAb of the invention has modified expression, stability, half-life, antigen binding, heavy chain -light chain pairing, tissue penetration or a combination thereof as compared to a parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

Monoclonal Antibodies

In one embodiment, the synthetic antibody-like biologic comprises an intact monoclonal antibody, an immunologically active fragment (e.g., a Fab or (Fab)₂ fragment), a monoclonal antibody heavy chain, or a monoclonal antibody light chain.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can treat, prevent, and/or protect against disease, such as an infection or cancer, in the subject administered a composition of the invention. The antibody, by binding the antigen, can treat, prevent, and/or protect against disease in the subject administered the composition. The antibody can promote survival of the disease in the subject administered the composition. In one embodiment, the antibody can provide increased survival of the disease in the subject over the expected survival of a subject having the disease who has not been administered the antibody. In various embodiments, the antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the antibody can provide increased protection against the disease in the subject over the expected protection of a subject who has not been administered the antibody. In various embodiments, the antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

As described above, the antibody can be generated in the subject upon administration of the composition to the subject. The antibody may have a half-life within the subject. In some embodiments, the antibody may be modified to extend or shorten its half-life within the subject. Such modifications are described herein in more detail.

The composition comprising a soluble receptor mimic synthetic antibody-like biologic, or nucleic acid molecule encoding the same, can be administered to the subject. The composition can result in the generation of the synthetic antibody-like biologic in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours.

The compositions of the present invention can have features required of effective compositions such as being safe so that the composition does not cause illness or death; being protective against illness; and providing ease of administration, few side effects, biological stability, and low cost per dose.

### Nucleic Acid Molecules

In one embodiment, the present invention provides a composition comprising an isolated nucleic acid sequence encoding a peptide or protein described herein. For example, in one embodiment, the composition comprises an isolated nucleic acid molecule encoding a soluble receptor protein, a fragment of a peptide or protein, or an antibody-like fusion protein as described herein.

In some embodiments, the isolated nucleic acid sequence encodes a protein or peptide comprising an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18. In some embodiments, the isolated nucleic acid sequence encodes a fragment comprising at least 20%, 30%, 40%, 50%, 60%, 65%, 70%, 75%, 80%, 85%, 90%, 91%, 92% 93%, 94%, 95%, 96%, 97%, 98%, or 99% of the full length of a protein or peptide comprising an amino acid sequence of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

Further, the invention encompasses an isolated nucleic acid encoding a polypeptide having substantial homology to a protein or peptide disclosed herein. In some embodiments, the isolated nucleic acid sequence encodes protein or peptide having at least 75%, 80%, 85%, 90%, 91%, 92% 93%, 94%, 95%, 96%, 97%, 98%, or 99% sequence identity with SEQ ID NO: 2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18.

In some embodiments, the isolated nucleic acid sequence comprises a nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In some embodiments, the isolated nucleic acid sequence comprises a fragment comprising at least 20%, 30%, 40%, 50%, 60%, 65%, 70%, 75%, 80%, 85%, 90%, 91%, 92% 93%, 94%, 95%, 96%, 97%, 98%, or 99% of the full length of a nucleotide sequence of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

In one embodiment, the nucleic acid molecule comprises a nucleotide sequence having substantial homology to SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. In some embodiments, the isolated nucleic acid sequence has at least 75%, 80%, 85%, 90%, 91%, 92% 93%, 94%, 95%, 96%, 97%, 98%, or 99% sequence identity to SEQ ID NO: 1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

The isolated nucleic acid sequence can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA, cDNA, and RNA. For example, in one embodiment, the composition comprises an isolated DNA molecule, including for example, an isolated cDNA molecule, encoding a protein inhibitor or functional fragment thereof. In one embodiment, the composition comprises an isolated RNA molecule encoding a protein inhibitor or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. The above modifications may be combined.

In some instances, the nucleic acid molecule comprises at least one of the following chemical modifications: 2′-H, 2′-O-methyl, or 2′-OH modification of one or more nucleotides. In some embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target. In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′ dimethylaminopropyl (2′-O-DMAP), 2′-O-dimethylaminoethyloxyethyl (2′ DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

Therefore, in another aspect, the invention relates to a vector, comprising the nucleotide sequence of the invention or the construct of the invention. The choice of the vector will depend on the host cell in which it is to be subsequently introduced. In some embodiments, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

In some embodiments, the expression of synthetic nucleic acids encoding a protein is typically achieved by operably linking a nucleic acid encoding the protein or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and an IgE signal peptide.

The vectors of the present invention may also be used for nucleic acid immunization, using standard gene delivery protocols. Methods for gene delivery are known in the art. See, e.g., U.S. Pat. Nos. 5,399,346, 5,580,859, 5,589,466, incorporated by reference herein in their entireties.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or protein of invention, described elsewhere herein.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In some embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of transformed or transfected host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a protein inhibitor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a peptide or protein into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a peptide or protein of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a peptide or protein into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments, that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular polypeptide, e.g., by immunological means (ELISAs and Western blots) or by assays described herein to identify agents falling within the scope of the invention.

### Delivery Vehicles

In one embodiment, the present invention provides a composition comprising delivery vehicle comprising a synthetic soluble receptor mimic polypeptide, or nucleic acid molecule encoding the same, as described herein.

Exemplary delivery vehicles include, but are not limited to, microspheres, microparticles, nanoparticles, polymerosomes, liposomes, and micelles. For example, in some embodiments, the delivery vehicle is loaded with synthetic soluble receptor mimic polypeptide, or a nucleic acid molecule encoding a synthetic soluble receptor mimic polypeptide. In some embodiments, the delivery vehicle provides for controlled release, delayed release, or continual release of its loaded cargo. In some embodiments, the delivery vehicle comprises a targeting moiety that targets the delivery vehicle to a treatment site.

### Substrates

In one embodiment, the present invention provides a scaffold, substrate, or device comprising a synthetic soluble receptor mimic polypeptide, or nucleic acid molecule encoding the same, as described herein.

For example, in some embodiments, the present invention provides a tissue engineering scaffold, including but not limited to, a hydrogel, electrospun scaffold, polymeric matrix, or the like, comprising the modulator. In certain embodiments, the a synthetic soluble receptor mimic polypeptide, or nucleic acid molecule encoding the same as described herein, may be coated along the surface of the scaffold, substrate, or device. In certain embodiments, the modulator is encapsulated within the scaffold, substrate, or device.

### Multimeric Compositions

In one embodiment, the composition comprises a multimeric synthetic antibody-like biologic, for example a dimeric or trimeric synthetic antibody-like biologic. In one embodiment, the composition comprises two or more soluble receptor mimics which dimerize, trimerize, or form higher order multimeric complexes. In one embodiment, the two or more soluble receptor mimics are mimics of the same receptor, for example a homotrimer of mimics of the Ace2 receptor. In one embodiment, the two or more soluble receptor mimics are mimics of one or more different receptors, for example a heterodimer of an Ace2 receptor mimic and a CD147 mimic.

In one embodiment, two or more synthetic antibody-like reporter mimics which associate to form a homomeric or heteromeric composition are encoded on a single nucleic acid molecule. In one embodiment, the invention relates to a combination of a first nucleic acid molecule encoding a first synthetic antibody-like reporter mimic and a second nucleic acid molecule encoding a second synthetic antibody-like reporter mimic.

### Pharmaceutical Compositions

The present invention also provides pharmaceutical compositions comprising one or more of the compositions described herein. Formulations may be employed in admixtures with conventional excipients, i.e., pharmaceutically acceptable organic or inorganic carrier substances suitable for administration to a treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group: benzyl alcohol, sorbic acid, parabens, imidurea and combinations thereof.

In one embodiment, the composition includes an anti-oxidant and a chelating agent that inhibits the degradation of one or more components of the composition. Exemplary antioxidants for some compounds are BHT, BHA, alpha-tocopherol and ascorbic acid. Exemplary chelating agents include edetate salts (e.g. disodium edetate) and citric acid. The chelating agent is useful for chelating metal ions in the composition that may be detrimental to the shelf life of the formulation. While BHT and disodium edetate may be the antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the compounds or other compositions of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para hydroxybenzoates, ascorbic acid, and sorbic acid.

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, chewing gum, varnishes, sealants, oral and teeth “dissolving strips”, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Treatment Methods

The present invention provides a method for the treatment or prevention of a viral disease or disorder which requires interaction with a cellular receptor protein as an essential step in its life cycle. The present method may be used to treat or prevent a coronavirus disease or disorder. In some embodiments, the method may be used to treat or prevent SARS or COVID-19.

In one aspect, the invention provides a method for preventing in a subject, a disease or disorder, by administering to the subject a composition described herein. Administration of a prophylactic agent can occur prior to the manifestation of symptoms characteristic of the disease or disorder, such that the disease or disorder is prevented or delayed in its progression.

In some embodiments, the method comprises administering an effective amount of a composition described herein to a subject diagnosed with, suspected of having, or at risk for developing a viral disease or disorder which requires interaction with a cellular receptor protein as an essential step in its life cycle. In one embodiment, the composition is administered systemically to the subject.

Also provided herein is a method of treating, protecting against, and/or preventing disease in a subject in need thereof by administering a combination of a soluble receptor mimic of the invention and a composition for generating a synthetic antibody in the subject. In some embodiments, the method can include administering a single composition comprising a soluble receptor mimic of the invention and a synthetic antibody, or recombinant nucleic acid molecule encoding the same, to the subject. In some embodiments, the method can include administering a combination of a first composition comprising a soluble receptor mimic of the invention and second composition comprising a synthetic antibody, or recombinant nucleic acid molecule encoding the same, to the subject. In some embodiments, a synthetic antibody administered in combination with the soluble receptor mimic of the invention is a neutralizing antibody, such as the SARS/CoV2 neutralizing antibody CR3022 scFv-Fc.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are administered by i.v. injection.

The pharmaceutical compositions useful for practicing the invention may be administered to deliver a dose of from 1 ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage will vary from about 1 μg to about 50 mg per kilogram of body weight of the mammal. In one embodiment, the dosage will vary from about 1 mg to about 10 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Mimicking Soluble Receptors to Trick and Neutralize SARS-CoV2 Virus

The experiments described herein demonstrate the creation and use of soluble decoys receptors. Unlike neutralizing antibodies, viruses cannot mutate to avoid detection to soluble receptors because recognition of cellular receptors is a required step in the viral lifecycle. SARS-CoV2 virus is thought to enter cells through attachment to the ACE2 receptor. Indeed, the experiments provided herein demonstrate that SARS-CoV2 has high affinity for soluble ACE2. In addition, there is evidence that CD147 may be an alternative receptor for SARS-CoV2. Lastly, without wishing to be bound by any particular theory, CD107a plays a role in SARS entry and may also interact with SARS-CoV2. Therefore, experiments were designed to engineer high affinity, multivalent therapeutics encoding minimal versions of these soluble receptors.

### Structure-Based Design of Soluble Receptor Immunotherapeutics.

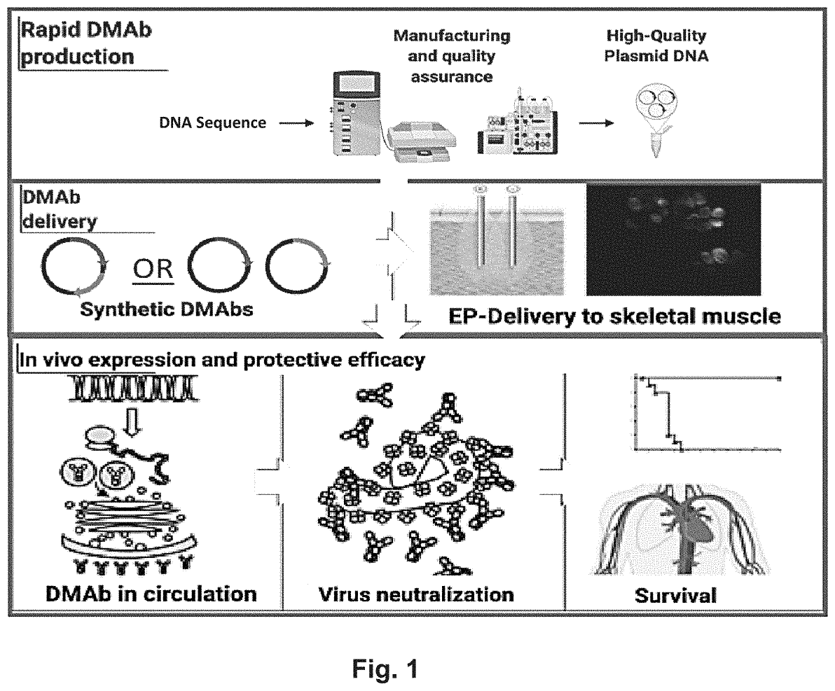

A multi-pronged strategy was used to design minimal soluble decoys amenable for use with DNA delivery methods (FIG. 1): 1) Rapid structure-based computational design simulations to create stabilized and/or minimal domains encoding the ‘business end’ of soluble receptors, optimize protein tertiary motifs and secondary structures, proline-stabilized loops and engineer disulfide bonds. 2) Optimize potency by employing saturated mutagenesis at interface residues. 3) Re-design surfaces of biotherapeutics in a way to limit cross-recognition to the matching cellular receptors.

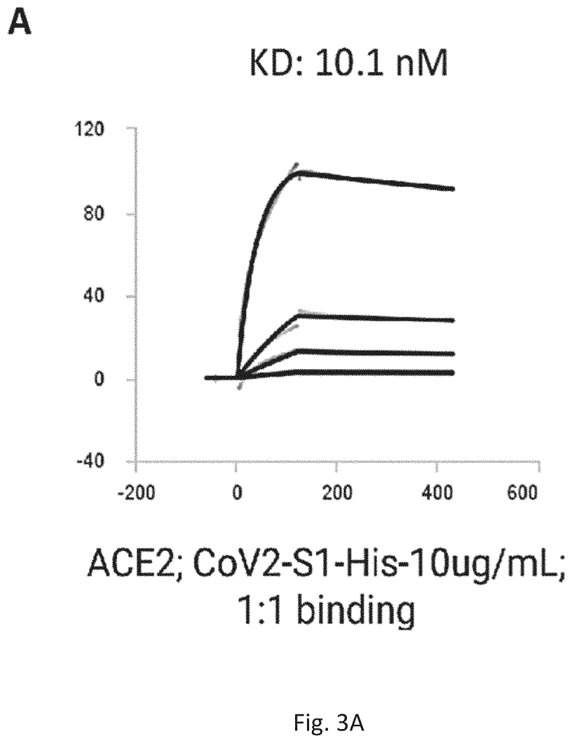

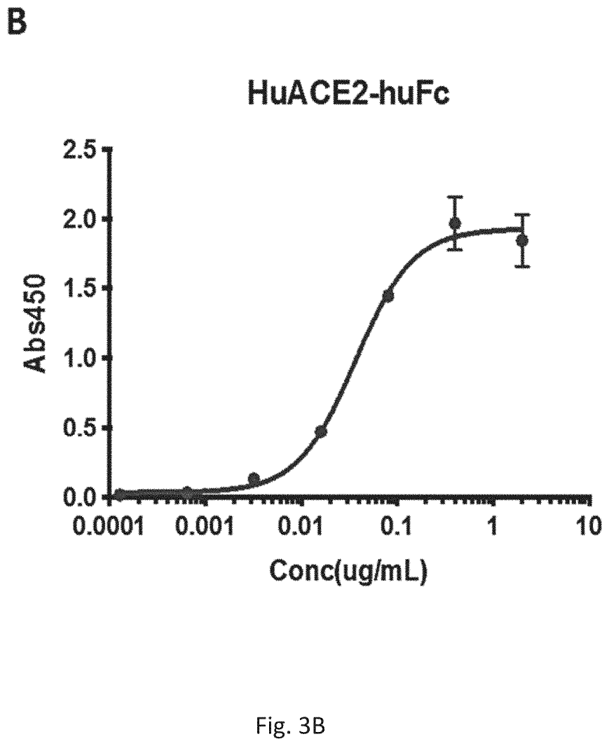

Using the molecular design platform, ROSETTA and MSL, fully glycosylated models of CoV2 spike have been made using cryo-EM and crystal structures. With starting structures in hand, stabilizing mutations are designed into the CoV2 Spike. Computational resurfacing and glycan resurfacing is used to change immune recognition of the antigens. Using these methods, a second round of design is employed for those constructs with significant molecular surface in common with the membrane-bound host cellular receptors (distal to the paratope). Resulting computationally designed variants are selected based on ROSETTA score and structural metrics, ensuring the models are consistent with well-folded natural proteins. Selected designs are rapidly synthesized using the BioXp DNA printer and produced in a high throughput assay using suspension mammalian cell lines (Expi293) in order to produce an immunogen with mammalian glycans. This assay will assess expression and binding profiles. The constructs with strong expression and binding are produced at larger scale (1L Expi293 cells). The proteins are purified by Protein A affinity columns and size-exclusion chromatography on an automated Akta Pure systems. The stability is determined by SYRPO orange/qPCR. The binding profile is determined using ELISA using anti-His capture of SARS-CoV2 spike proteins (FIG. 3). SPR experiments are also employed on a Biacore 8k using a BiotinCAP chip for reversible capture of biotinylated SARS-CoV2 spike proteins. The focus of this proposal is on Ace2 and CD147, but CD107a is also being tested for affinity to SARS-CoV2.

Synthetic DMAB Engineering for In Vivo Delivery of Covid-19 Therapeutics.

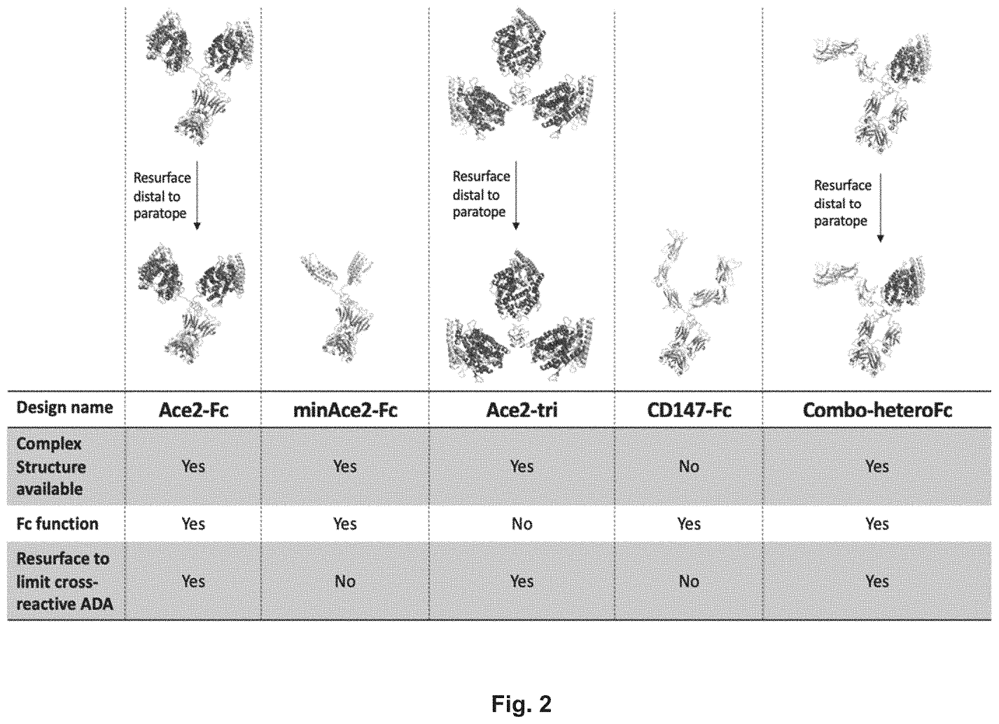

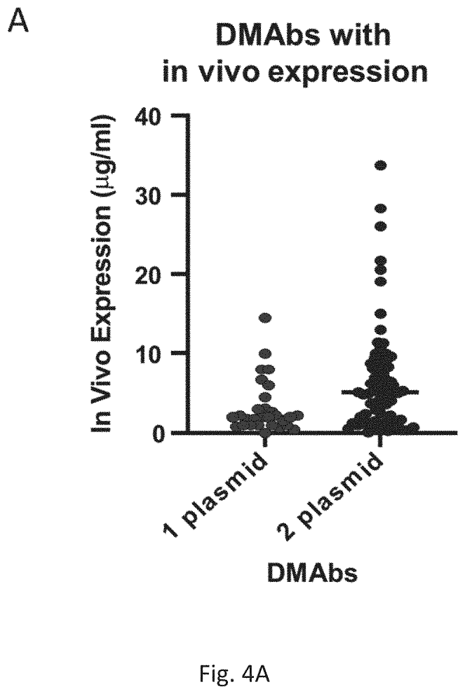

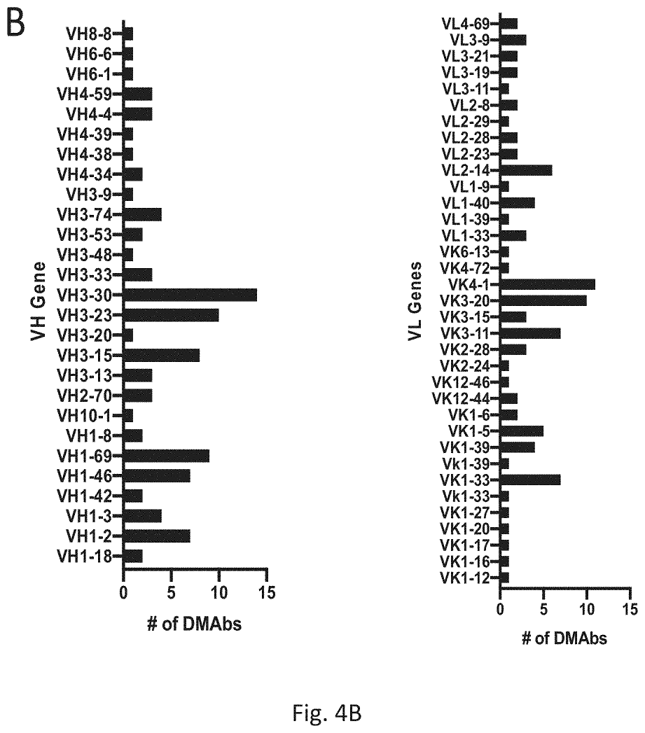

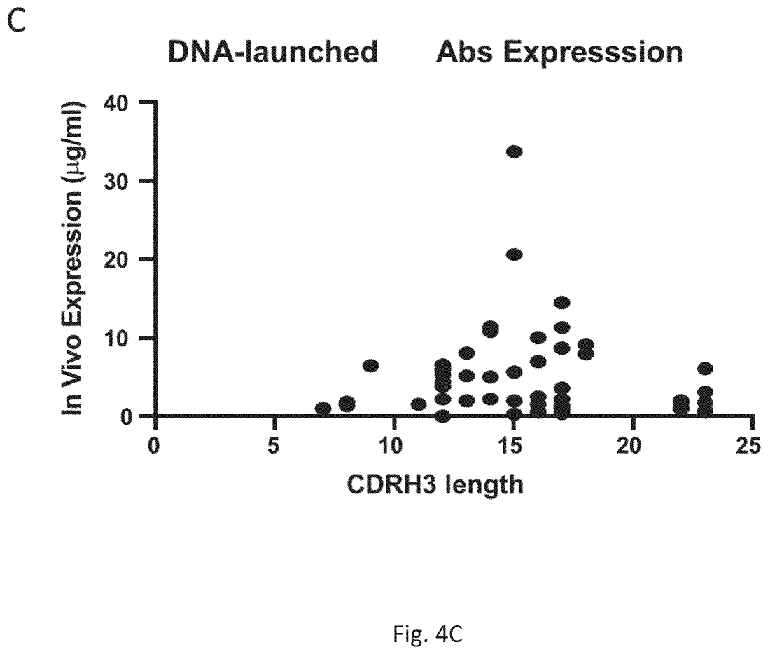

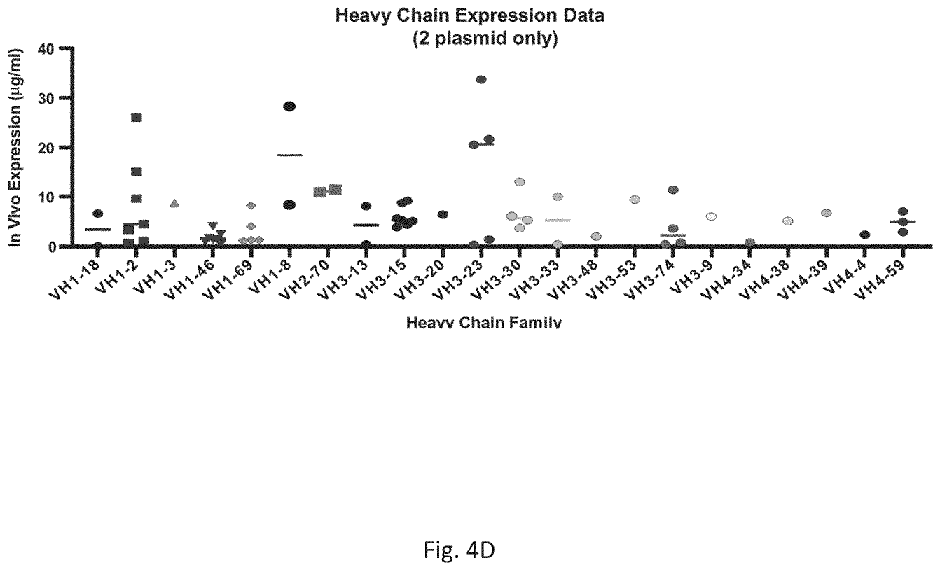

Datasets of in vivo produced antibodies using nucleic acids have been built, and a wide variety of variable heavy and light chain antibody families have been delivered. The engineered soluble decoy receptors with the strongest in vitro data are fused to antibody Fc domains to create an antibody-like biologic. The Fc fusions are optimized to enhance in vivo expression, half-life extension to prolong DMAb expression for several months, and to enhance functional activity. In vivo expression studies of engineered receptor molecules for ACE2, CD147 and CD107a are pursued in BALB/c and B6 mice (FIG. 4). In vivo pharmacokinetic assays in mice are performed to study magnitude and duration of expression. ELISA and SPR binding assays are employed at multiple time points after DMAb delivery. Pseudoviral neutralization assays are performed to determine functionality of in vivo produced DMAbs compared to recombinant ACE2/CD147/CD107a receptors. Next, receptor designs are combined into a single therapeutic using optimized knob/hole Fc mutations which enable efficient heterodimer formation. (heteroFc in FIG. 2). In addition, SARS/CoV2 neutralizing antibody CR3022 is also used as an scFv-Fc partner for the soluble mimics. Each of the Combo-heteroFc designs is tested for expression in BALB/c mice.

### Evaluation of Expression and Functionality in Pigs.

The Pig model is highly relevant because the body size and metabolism are more similar to humans than other preclinical models. DMAb expression is assessed in pigs prior to going into the clinic. DMAbs are subjected to dosing studies in outbred 8-week old Landrace pigs (or similar) at either 500 μg or 1 mg (n=3, 18 pigs total). The pigs are administered DMAbs three times at 3-week intervals. The pigs are injected with a split dose into quadriceps and trapezius muscles. The 3P device has been used in pigs previously to delivery DNA.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a nucleic acid molecule encoding at least one synthetic cellular receptor peptide which specifically binds to a coronavirus Spike antigen.

2. The composition of claim 1, wherein the synthetic cellular receptor peptide is selected from the group consisting of ACE2, CD147, CD107a, CD13, GRP78 and DPP4, a fragment thereof, and a variant thereof.

3. The composition of claim 1, wherein the synthetic cellular receptor peptide is fused to an antibody Fc domain.

4. The composition of claim 1, wherein two or more synthetic cellular receptor peptides form a multimeric complex.

5. The composition of claim 1, wherein the nucleic acid molecule comprises a nucleotide sequence selected from the group consisting of:
(a) the nucleotide sequence encodes a peptide comprising an amino acid sequence having at least about 90% identity over an entire length of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18;
(b) the nucleotide sequence encodes a peptide fragment comprising at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18; and
(c) the nucleotide sequence encodes a peptide comprising an amino acid sequence having at least about 90% identity to at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

6. The composition of claim 1, wherein the nucleic acid molecule encodes an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

7. The composition of claim 1, wherein the nucleic acid molecule comprises a nucleotide sequence selected from the group consisting of:
a) the nucleotide sequence having at least about 90% identity over an entire length of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17;
b) the nucleotide sequence comprises at least 30% of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17; and
c) the nucleotide sequence comprises at least about 90% identity to at least 30% of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17.

8. The composition of claim 1, wherein the nucleic acid molecule comprises the nucleic acid sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17.

9. The composition of claim 1, wherein the nucleic acid molecule comprises an expression vector.

10. The composition of claim 1, wherein the nucleic acid molecule is incorporated into a viral particle.

11. The composition of claim 1, further comprising a pharmaceutically acceptable excipient.

12. The composition of claim 1, further comprising an adjuvant.

13. A composition comprising at least one synthetic cellular receptor peptide which specifically binds to a coronavirus Spike antigen.

14. The composition of claim 13, wherein the synthetic cellular receptor peptide is selected from the group consisting of ACE2, CD147, CD107a, CD13, GRP78 and DPP4, a fragment thereof, and a variant thereof

15. The composition of claim 13, wherein the synthetic cellular receptor peptide is fused to an antibody Fc domain.

16. The composition of claim 13, wherein two or more synthetic cellular receptor peptides form a multimeric complex.

17. The composition of claim 13, wherein the synthetic cellular receptor peptide comprises an amino acid sequence selected from the group consisting of:
a) the peptide comprising an amino acid sequence having at least about 90% identity over an entire length of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18;
b) the peptide fragment comprising at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18; and
c) the peptide fragment comprising an amino acid sequence having at least about 90% identity to at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

18. The composition of claim 13, wherein the synthetic cellular receptor peptide comprises an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

19. The composition of claim 13, further comprising a pharmaceutically acceptable excipient.

20. The composition of claim 13, further comprising an adjuvant.

21. A nucleic acid molecule comprising a nucleotide sequence encoding at least one synthetic cellular receptor peptide which specifically binds to a coronavirus Spike antigen.

22. The nucleic acid molecule of claim 21, wherein the synthetic cellular receptor peptide is selected from the group consisting of ACE2, CD147, CD107a, CD13, GRP78 and DPP4, a fragment thereof, and a variant thereof.

23. The nucleic acid molecule of claim 21, wherein the synthetic cellular receptor peptide is fused to an antibody Fc domain.

24. The nucleic acid molecule of claim 21, wherein two or more synthetic cellular receptor peptides form a multimeric complex.

25. The nucleic acid molecule of claim 21, wherein the nucleic acid molecule comprises a nucleotide sequence selected from the group consisting of:
a) the nucleotide sequence encodes a peptide comprising an amino acid sequence having at least about 90% identity over an entire length of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18;
b) the nucleotide sequence encodes a peptide fragment comprising at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18; and
c) the nucleotide sequence encodes a peptide comprising an amino acid sequence having at least about 90% identity to at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

26. The nucleic acid molecule of claim 21, wherein the nucleic acid molecule encodes an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

27. The nucleic acid molecule of claim 21, wherein the nucleic acid molecule comprises a nucleotide sequence selected from the group consisting of:
a) the nucleotide sequence having at least about 90% identity over an entire length of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17;
b) the nucleotide sequence comprises at least 30% of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17; and
c) the nucleotide sequence comprises at least about 90% identity to at least 30% of the nucleotide sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17.

28. The nucleic acid molecule of claim 21, wherein the nucleic acid molecule comprises the nucleic acid sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17.

29. The c nucleic acid molecule of claim 21, wherein the nucleic acid molecule comprises an expression vector.

30. The nucleic acid molecule of claim 21, wherein the nucleic acid molecule is incorporated into a viral particle.

31. A synthetic cellular receptor peptide which specifically binds to a coronavirus Spike antigen.

32. The peptide of claim 31, wherein the synthetic cellular receptor peptide is selected from the group consisting of ACE2, CD147, CD107a, CD13, GRP78 and DPP4, a fragment thereof, and a variant thereof.

33. The peptide of claim 31, wherein the synthetic cellular receptor peptide is fused to an antibody Fc domain.

34. The peptide of claim 31, wherein two or more synthetic cellular receptor peptides form a multimeric complex.

35. The peptide of claim 31, wherein the synthetic cellular receptor peptide comprises an amino acid sequence selected from the group consisting of:
a) the peptide comprising an amino acid sequence having at least about 90% identity over an entire length of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18;
b) the peptide fragment comprising at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18; and
c) the peptide fragment comprising an amino acid sequence having at least about 90% identity to at least 30% of the amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

36. The peptide of claim 31, wherein the synthetic cellular receptor peptide comprises an amino acid sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18.

37. A method of treating or preventing a disease or disorder associated with a coronavirus infection in a subject in need thereof, the method comprising administering a composition of any one of claim 1-20, a nucleic acid molecule of any one of claim 21-30 or a peptide of any one of claim 31-36 to the subject.

38. The method of claim 37, wherein the disease or disorder associated with a coronavirus is selected from the group consisting of SARS, 229E, NL63, OC43, HKU1, MERS and SARS-CoV-2 (COVID-19).

39. The method of claim 37, wherein administering includes at least one of electroporation and injection.

40. A method of protecting a subject in need thereof from infection with a coronavirus, the method comprising administering a composition of any one of claim 1-20, a nucleic acid molecule of any one of claim 21-30 or a peptide of any one of claim 31-36 to the subject.

41. The method of claim 40, wherein the coronavirus is selected from the group consisting of SARS, 229E, NL63, OC43, HKU1, MERS and SARS-CoV-2 (COVID-19).

42. The method of claim 37, wherein administering includes at least one of electroporation and injection.
